(19) United States
(12) Patent Application Publication
(10) Pub. No.: US 2025/0197814 A1
(43) Pub. Date: Jun. 19, 2025

(54) METHOD FOR PRESERVING CELLS

(71) Applicants: SORBONNE UNIVERSITE, Paris (FR); CENTRE NATIONAL DE LA RECHERCHE SCIENTIFIQUE, Paris (FR)

(72) Inventors: Francisco FERNANDES, Paris (FR); Christophe HELARY, Paris (FR); Kankan QIN, Paris (FR)

(21) Appl. No.: 18/849,747

(22) PCT Filed: Mar. 22, 2022
(86) PCT No.: PCT/FR2022/050530

§ 371 Date: Sep. 23, 2024

(51) Int. Cl. C12N 5/00

(52) U.S. Cl. CPC C12N 5/525; C12N 5/562

(57) ABSTRACT

The present disclosure relates to a method for preserving free cells, such as blood cells, by freezing in the presence of a nontoxic cryoprotectant, i.e., one that can be administered to a living human or animal organism. The present disclosure thus relates to methods, compositions and kits for freezing and storing non-adherent cells, such as red blood cells, for extended periods of time while preventing the lesions that can occur during the storage thereof and, in the case of red blood cells, while preserving deformability and increasing survival.

### CROSS-REFERENCE TO RELATED APPLICATION

This present application is a national stage application of International Patent Application No. PCT/FR2022/050530, filed Mar. 22, 2022, the disclosure of which is hereby incorporated by reference in its entirety.

### TECHNICAL FIELD

The present disclosure relates to a method for preserving free cells, such as blood cells, by freezing in the presence of a non-toxic cryoprotectant, i.e. one that can be administered to a living human or animal organism.

The present disclosure thus relates to methods, compositions and kits for freezing and storing non-adherent cells, such as red blood cells, for extended periods of time while preventing the lesions that may occur during the storage thereof and, in the case of red blood cells, while preserving deformability and increasing survival.

### BACKGROUND

In this context, and for example, blood transfusion is widely used for medical purposes, in particular for cardiovascular surgical care, severe burns, transplant surgery, massive trauma, pregnancy-related complications and therapy of solid and hematological malignant tumors.

Generally, blood intended for transfusion is preserved for up to 42 days at 4° C. However, blood preserved at this temperature for more than 21 days is considered old blood, in which depletion of metabolites, loss of cell membrane integrity, immune mediators, and hemolysis may be observed.

The use of anticoagulants and suitable preservation media has made it possible to limit the damage caused to red blood cells during their storage and to increase the safety and clinical efficiency of allogeneic or autologous blood transfusions.

An alternative to this preservation method is freezing; freezing is of particular interest in particular for military use and the storage of blood cells for the treatment of rare diseases. There are two methods generally used for the cryopreservation of red blood cells:
the slow cooling technique (approximately −1° C./min) in a medium with a high glycerol content (approximately 40%) and allows preservation at −80° C.; and
the rapid cooling technique (greater than −100° C./min) in a medium with a low glycerol content (approximately 20%) and allows preservation at −196° C.

However, excessive amounts of glycerol lead to osmotic imbalance problems, which can cause hemolysis. To avoid this, clinical staff must perform complex and time-consuming deglycerolization operations after thawing, including the preparation of different concentrations of washing solution, multiple centrifugations, repeated elimination of used supernatant liquid.

Glycerol, like ethylene glycol, DMSO or DMF, used as a cryoprotectant protects red blood cells by limiting the formation of ice crystals in the intracellular space, decreasing the exposure of cells to excessive electrolyte concentrations during freezing and preventing excessive contraction of the cytoplasmic volume.

There are other extracellular cryoprotectants that can be used for cryopreservation of red blood cells, including, for example, hydroxyethyl starch, polyvinylpyrrolidone (PVP), and dextran, which have the main advantage of not crossing the cell membrane. Although these non-permeable cryoprotectants are biodegradable and tolerated by the human organism, it is still necessary to eliminate them after thawing.

Finally, cryoprotectants such as trehalose, whose cryoprotective action in living systems is known, do not spontaneously cross the cell membrane but have a predominant action in the intracellular space, are often used in the presence of adjuvants allowing its transport such as hydroxyapatite particles or in modified forms such as acetylated trehalose.

However, it is still necessary to optimize the cryopreservation of cells; this is what the Applicant has achieved by developing a method for cryopreserving cells that does not require toxic cryopreservation agents to be eliminated after thawing and also has the advantage of being able to be implemented with mechanical freezers.

### SUMMARY

The present disclosure thus relates to an original method for preserving non-adherent cells by directional freezing comprising the steps of:
a) suspending said non-adherent cells, at a concentration that may be between 1×10⁶ and 5×10⁹ cells/mL, preferably the cell concentration is between 1×10⁸ and 5×10⁹ cells/mL, in a cryopreservation medium composed of an isotonic aqueous solution, preferably saline, for example a 0.9% by weight aqueous solution of NaCl, and at least one non-toxic cryoprotectant, preferably at a concentration of between 4 and 50% by weight/volume, preferably this cryoprotectant is a natural protein, preferably natural proteins with a molecular weight of between 10 and 80 kDa, and may be selected from albumins, alpha and beta globulins and fibrinogen; optionally, the mixture may also comprise a cryoprotective additive such as carboxymethyl cellulose, sucrose, trehalose or glucose;
b) optionally, conditioning the mixture obtained in step a);
c) directional freezing of the mixture obtained in step a);
d) preservation at a temperature between −79° C. and −197° C., preferably at approximately-80° C.

This method allows the preservation of any so-called non-adherent or free cell, i.e. circulating cells or adherent cells temporarily in suspension, i.e. detached from their physiological support/environment; in particular, these cells are selected from:
Blood cells (red blood cells, white blood cells, platelets);
Hepatocytes;
Neuronal cells;
Rare cells (linked to particular pathologies) whose preservation could be of future interest;
Immunomodulatory cells (Tregs, etc.);
Stem cells, iPSCs.

Preferably, these are blood cells and even more preferably red blood cells.

Prior to the implementation of step a), it may be necessary to separate the cells from their medium; this is in particular the case for blood cells present in a blood sample that should be separated from the plasma; this is carried out using conventional techniques known to those skilled in the art, such as centrifugation for example.

The cryoprotectant is non-toxic, i.e. it can be administered to a human or animal patient; it is therefore not essential to purify the medium containing the cells after they have been thawed; the cryoprotectant is a natural protein that can be selected from:
Human or bovine albumin, ovalbumin;
Alpha-1 glycoprotein, fibrinogen, . . .

Preferably, when the cells preserved by the method according to the disclosure are intended to be administered to a human, the cryoprotectant is human serum albumin (also called HSA).

Preferably, serum albumin is present in a cryopreservation medium at a concentration of between 4 and 50% by weight/volume, more preferably between 20 and 50% by weight/volume.

According to one embodiment, the cryopreservation medium consists of an isotonic aqueous solution and at least one of the aforementioned cryoprotectants.

According to another embodiment, the cryopreservation medium consists of an isotonic aqueous solution, at least one of the aforementioned cryoprotectants and at least one cryoprotective additive selected from sucrose, glucose, trehalose and carboxymethyl cellulose.

According to this embodiment, carboxymethyl cellulose can be used at a concentration of between 0.1 and 1% w/v; trehalose at a concentration of between 0.5 and 5% w/v and sucrose at a concentration of between 0.5 and 5% w/v; glucose has a concentration of between 0.5 and 5% w/v.

Directional freezing is a freezing technique that consists in linearly moving a sample from a thermalized support at a temperature higher than the melting temperature of the sample (called a hot block) to a thermalized support at a temperature lower than the melting temperature of the sample (called a cold block). The distance between the two supports as well as the temperatures of each of the supports can be independently selected to achieve a controlled thermal gradient. The temperature range of the hot block is between 0.5 and 40° C. and the temperature range of the cold block is between −60 and −100° C. The speed at which the sample, placed in the freezing chamber, and moved between the hot block and the cold block corresponds to the growth rate of the ice front and is controlled by a dedicated motor that operates between 10 and 200 μm/s. For example, directional freezing can be implemented according to the method described in Qin et al., 2020.

The preservation temperature depends on the device used for preserving frozen cells; for example, it could be in the range of −80° C. with a variation of +/−3° C. in a mechanical freezer, in the range of −150° C. with a variation of +/−3° C. in a freezer capable of producing lower temperatures, in the range of −196° C. if the preservation is carried out in liquid nitrogen and between −135° C. and −190° C. if the preservation is carried out in nitrogen vapor.

The present disclosure also relates to a method for thawing suspended cells preserved according to the preservation method of the disclosure.

This thawing method is thus preferably implemented after the preservation method according to the disclosure; it comprises the steps of:
i. contacting the freezing chamber containing frozen cells with a surface heated to a temperature of between 35 and 45° C. for between 10 and 40 s, until the ice crystals have completely melted;
ii. centrifugation;
iii. suspending the cells from the centrifugation supernatant in an isotonic aqueous solution comprising between 1 and 10% by weight/volume of proline.

The present disclosure further relates to a composition comprising, or consisting of, non-adherent cells and an isotonic aqueous solution, preferably saline, comprising at least one non-toxic cryoprotectant at a concentration of between 4 and 50% by weight/volume and selected from albumins, alpha and beta globulins and fibrinogen. Optionally, the composition may also comprise a cryoprotective additive such as sucrose, trehalose, glucose or carboxymethyl cellulose; in this embodiment, carboxymethyl cellulose may be used at a concentration of between 0.1 and 1% w/v; trehalose at a concentration of between 0.5 and 5% w/v, sucrose at a concentration of between 0.5 and 5% w/v and glucose at a concentration of between 0.5 and 5% w/v.

Preferably, the cryoprotectant is human serum albumin.

The present disclosure further relates to a kit for preserving non-adherent cells comprising at least one sealed device for storing sterile biological fluids, which is flexible at room temperature and capable of withstanding without damage storage temperatures down to −196° C. and an isotonic aqueous solution, preferably saline, comprising at least one non-toxic cryoprotectant at a concentration of between 4 and 50% by weight and selected from albumins, alpha and beta globulins and fibrinogen and, optionally, a cryoprotective additive such as sucrose, trehalose, glucose and carboxymethyl cellulose, while preferably carboxymethyl cellulose may be used at a concentration of between 0.1 and 1% w/v, trehalose at a concentration of between 0.5 and 5% w/v, sucrose at a concentration of between 0.5 and 5% w/v and glucose at a concentration of between 0.5 and 5% w/v.

### BRIEF DESCRIPTION OF THE DRAWINGS

FIG. 1: Recovery of red blood cells (RBCs) after directional freezing with varying BSA volume fractions (BSA) (0.04, 0.14, and 0.28) at varying translation speeds. Upper panel: Image of centrifuged samples after the freezing-thawing process. The red color is due to hemoglobin released from the lysed cells. Neg, represents the negative control obtained without being frozen. Lower panel: Cell recovery after freezing and thawing.

FIG. 2: Cell recovery after directional freezing of RBCs (5×10⁹ cells·mL⁻¹) at 100 μm·s⁻¹·ns, indicates no significant difference.

FIGS. 3A-G: SEM and flow cytometry of RBCs after freezing and thawing. RBCs were stained with Calcein-AM for flow cytometry. Negative and positive controls indicate unstained and calcein-stained cells without undergoing freezing. Varying BSA volume fractions and ice front speeds were applied to the experimental groups. Scale bar, 2 μm.

FIGS. 4A-B: (A) Cell size (FSC-A) of the calcein-stained red blood cells. (B) Comparison of the cell size between the positive control (fresh cell) and the cryopreserved cells. Frozen cells with φBSA of 0.14 showed a significantly smaller size than fresh cells. ***, indicates P<0.01.

FIGS. 5A-B: Cell recovery (A) and flow cytometry (B) of RBCs after 106 days of freezer storage.

FIGS. 6A-B: (A) Cell size (FSC-A) of Calcein-stained RBCs after 106 days of freezer preservation at −80° C. (B) Comparison of the cell size between the positive control (fresh cell) and the cryopreserved cells. ***, indicates a significant difference at P<0.01.

FIG. 7: Efficiency of resuspension of thawed cells followed by freezing at 100 μm·s⁻¹. Positive control, absorbance of hemolysis in deionized H₂O with the same amount of cells compared to that in the presence of BSA.

FIGS. 8, 9, 10 and 11: Post-freezing cell suspension tests, histograms show the measurement of hemoglobin in the supernatant after suspension.

### DETAILED DESCRIPTION

### Examples

### Freezing Protocol

a. 5 mL of sheep blood in Alsever's solution (saline solution comprising 2.05% dextrose, 0.8% sodium citrate, 0.055% citric acid, and 0.42% sodium chloride) (1:1) is centrifuged at 600 g, 4° C. for 10 min;
b. The supernatant is removed and a 0.9% NaCl solution is added to restore the initial volume (5 mL);
c. The suspension obtained are centrifuged at 600 g, 4° C. for 10 min;
d. The supernatant is removed and a 0.9% NaCl solution is added to restore the initial volume (5 mL);
e. Steps c and d are repeated once more;
f. The concentration of red blood cells is calculated using a hemacytometer;
g. The cells necessary to obtain a concentration of 10⁸ or 1.5*10⁹ are transferred into 2 mL of BSA solution (between 4 and 50% by weight);
h. Cell suspensions are frozen in 500 μL CoverWell™ incubation chambers (Grace Bio-Labs) 22 mm×40 mm×0.5 mm on a thermal gradient of 50° C.·mm⁻¹ (passage from the thermal block at +10° C. to the thermal block at −90° C.; gap between blocks 2 mm). The translation speeds of the samples are 10, 50 and 100 μm·s⁻¹.
i. After freezing, the chambers containing the frozen cell suspensions are kept at −80° C. (from 1 day to 106 days).

### Thawing Protocol

a. The chambers containing the cells are thawed in contact with a surface at 45° C. for 10-15 s.
b. 200 μL of the contents of each chamber are transferred into an eppendorf, and centrifuged under the following conditions:
   1. For BSA concentrations less than or equal to 20%: 3000 rpm, 20° C., 10 min
   2. For BSA concentrations above 20%: 6000 rpm, 20° C., 20 min c. The supernatant is removed and the cells are suspended in a solution of 1% proline, 0.9% NaCl.
d. Steps b and c are repeated twice more for FACS characterization; four more times for SEM characterization.
e. The freezing-thawing efficiency is estimated from the measurement by UV-Vis absorption at 541 nm of the hemoglobin concentration in the supernatant obtained in step b.1 or b.2.

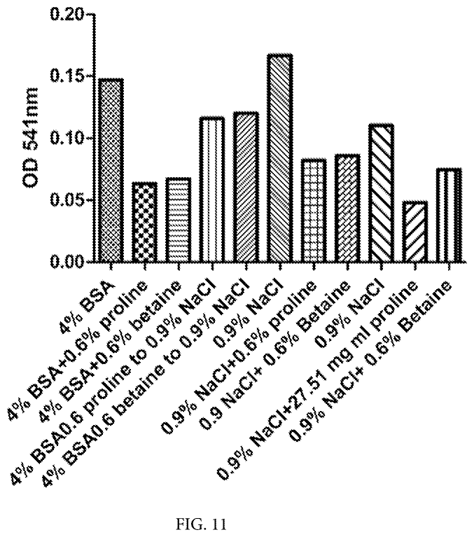

Other formulations were tested for post-thawing cell resuspension (results correspond to the measurement of hemoglobin in the supernatant after resuspension):
Proline (1, 2, 4, 8, 10%).
Proline (1, 2, 4, 8, 10%) then 4% BSA
(see FIG. 8)
4% BSA+proline (0.1, 0.5, 1, 2, 5%)
(see FIG. 9)
BSA+PBS
PBS
0.9% NaCl
BSA+glucose
BSA+glucose+trimethylglycine+proline
BSA+glucose+trimethylglycine+proline+glycine+glutamine
BSA+trimethylglycine+proline+glycine+glutamine
(see FIGS. 10 and 11 where C1: bsa+glucose; C2: C1+betaine+proline; C3: C2+glycine+glutamine; C4: C3-glucose)

1% Proline in 0.9% NaCl produces results similar to other proline concentrations, systematically higher than other formulations.

### Results

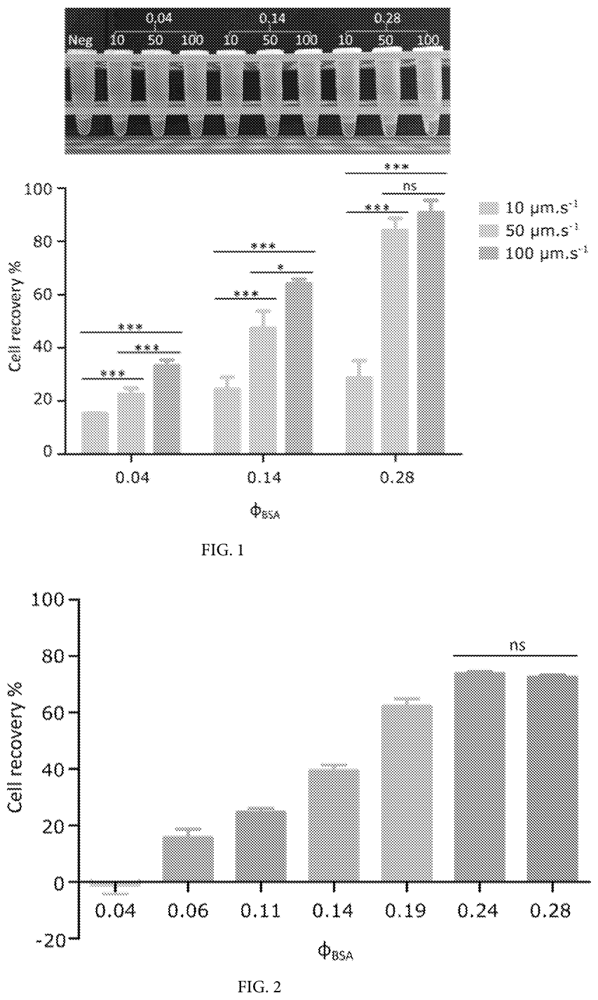

Increasing the BSA volume fraction (φBSA) resulted in improved cell recovery that was further improved by increasing the translation freezing speed implemented (FIG. 1).

For volume fractions of 0.04 to 0.28, cell recovery increased from ˜18% to ˜30% at 10 μm·s⁻¹ and from ˜38% to ˜95% at 100 μm·s⁻¹. For all volume fractions, increasing the translation speed resulted in better cell recovery, in particular for the φBSA=0.28 value that allows a three-fold increase when the translation speed varies from 10 to 100 μm·s⁻¹. After thawing and centrifugation, unlysed red blood cells (RBCs) were clustered at the bottom of the tube and hemoglobin from the broken cells was released into the supernatant, leading to a red color. For the negative control, the fresh blood sample showed almost no cell hemolysis, as shown by the colorless supernatant. Samples treated under other conditions showed a red color, except for φBSA between 0.28 and 50 at 100 μm·s⁻¹, confirming the positive role of highly concentrated BSA systems on recovery of RBCs.

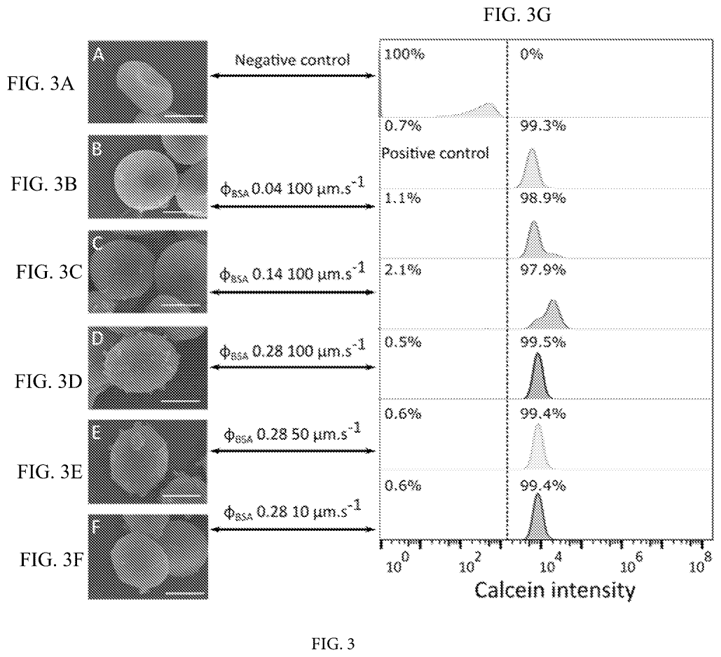

The cell density used in these experiments (10⁸ cells mL⁻¹) was 50 times lower than the physiological density (˜5×10⁹ cells mL⁻¹) of the human body. In the literature, the cell density has a positive effect that is essential for final cryosurvival. To account for this effect, another test was performed with a cell density of 5×10⁹ cells mL⁻¹ at 100 μm·s⁻¹ (FIG. 2). Frozen cells with a BSA volume fraction of 0.28 showed the highest recovery (˜80%), further confirming the beneficial effects of highly concentrated BSA on cell survival during directional freezing. However, a relatively lower survival compared to samples containing 10⁸ cells mL⁻¹ was observed, that can be attributed to the increase of mechanical stresses between cells.

### Quality of Red Blood Cells

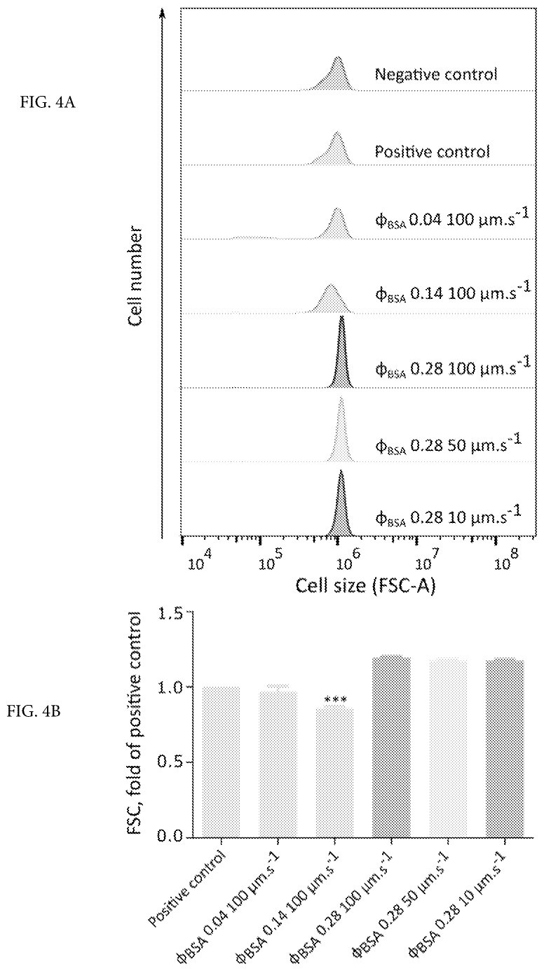

While the previous experiments allowed to determine the amount of erythrocytes whose membrane integrity was preserved, it is also useful to obtain additional information on the physiological state of the recovered cells. To this end, an observation by scanning electron microscopy and flow cytometry were carried out on the cells after freezing and thawing. It is known from the literature that healthy RBCs have a biconcave shape that gives them great flexibility and a high oxygen transport capacity; FIG. 3A shows that a majority of fresh RBCs have a biconcave disk shape. Cells frozen at 100 μm·s⁻¹ in the presence of a low BSA volume fraction (φBSA=0.04 and 0.14) showed a shape similar to that of fresh samples (FIGS. 3B-C).

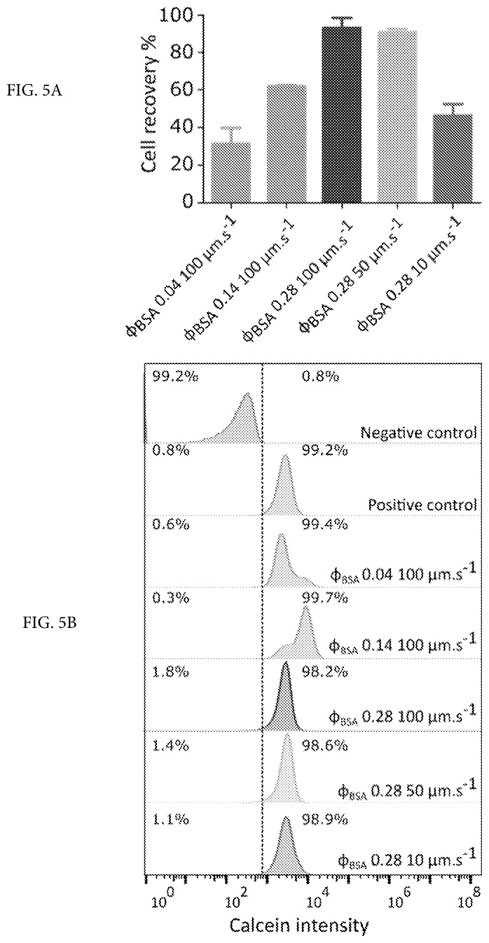

For the high-volume fraction groups, a majority of thawed cells have a disk shape in the absence of the concave feature, and a small portion of cells have morphological alteration toward a slightly spherical shape (FIG. 3D). Varying the translation speed did not significantly change the shape of RBCs when φBSA=0.28 (FIGS. 3E-F). Calcein-AM, a cell-permeable non-fluorescent dye that is converted to green fluorescent calcein by intracellular esterases in living cells, was used for the flow cytometry test. In FIG. 3G, the negative control (unstained fresh cells) and the positive control (stained fresh cells) have apparent distinct fluorescence intensity, which facilitates the determination of the experimental group. Regardless of the experimental conditions (BSA concentration and translation speed), more than 97% of the cells have a high calcein intensity. This indicates that, after resuspension, most of the recovered RBCs have esterase activity and are therefore alive. In other words, the slightly altered shape of RBCs does not affect their esterase activity. In flow cytometry analysis, “Forward scatter gating” is generally considered an index of cell size [9]. For the experimental groups, thawed RBCs showed a median cell size similar to that of the positive control, except those frozen in the presence of φBSA=0.14 (at 100 μm·s⁻¹) whose size is slightly smaller (FIGS. 4A-B). Considering both cell recovery and quality, more than 95% of RBCs have intact membranes with metabolic activities and similar cell size compared to the control after being cryopreserved via directional freezing with φBSA=0.28 at 100 μm·s⁻¹.

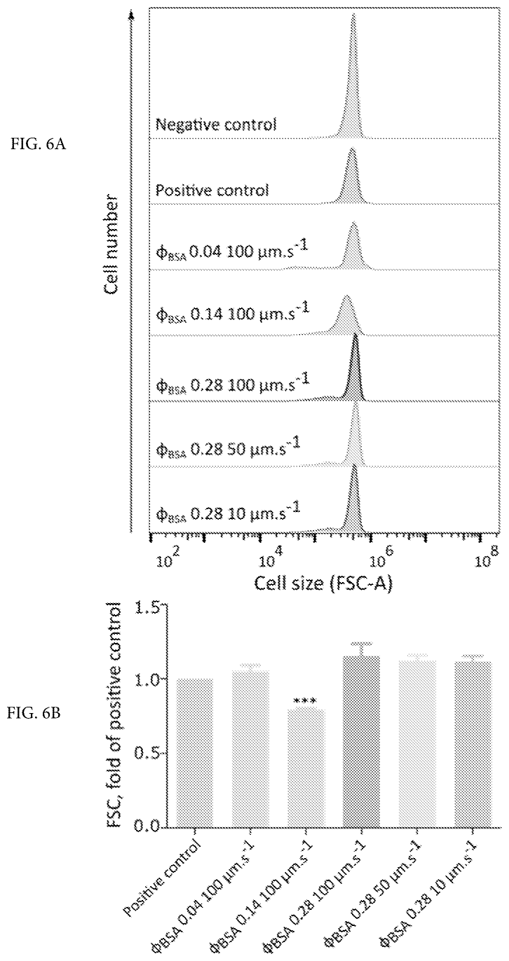

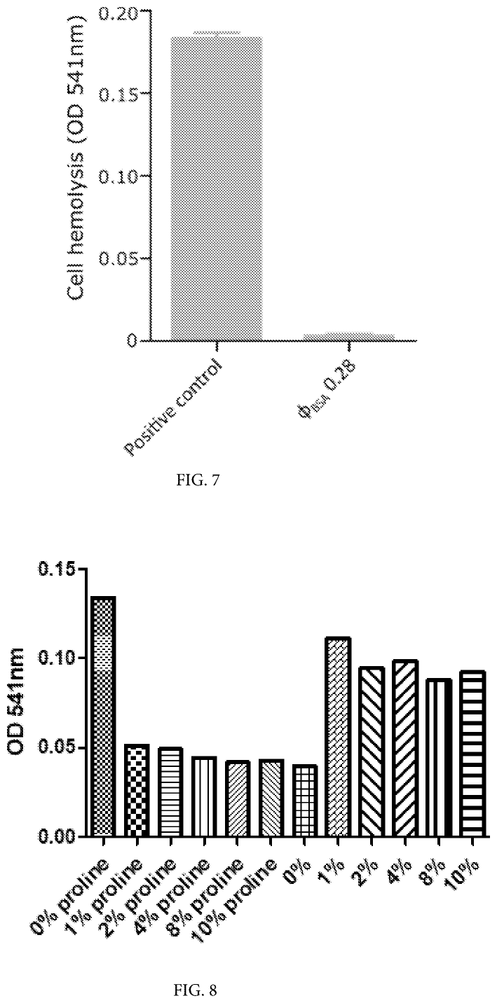

As previously described, the limited lifespan of RBCs, even in the presence of additives (<49 days), leads to a high demand for daily blood donors. Here, cryopreservation of RBCs was carried out by directional freezing, the RBCs were then stored in a freezer at −80° C. for three months (106 days). In FIG. 5A, cell recovery followed the same survival trend as that after one day of preservation. Red blood cell survival could be improved with an increase of BSA content and even more so with higher translation speed. After 106 days, samples prepared with a volume fraction of 0.28 at 100 μm·s⁻¹ showed ˜95% survival comparable to that of samples thawed after 1 day of preservation. Additional calcein staining (FIG. 5B) indicated that more than 98% of cells had esterase activity, regardless of freezing conditions. In all samples, the size of thawed cells was similar to that of the positive control, except for the group with a volume fraction of 0.14 that had a smaller size (FIG. 6). Thus, directional freezing in the presence of BSA (φBSA=0.28) allows twice as long preservation in the freezer without significantly affecting cell viability and vitality as the permitted shelf life of the red blood cells (49 days).

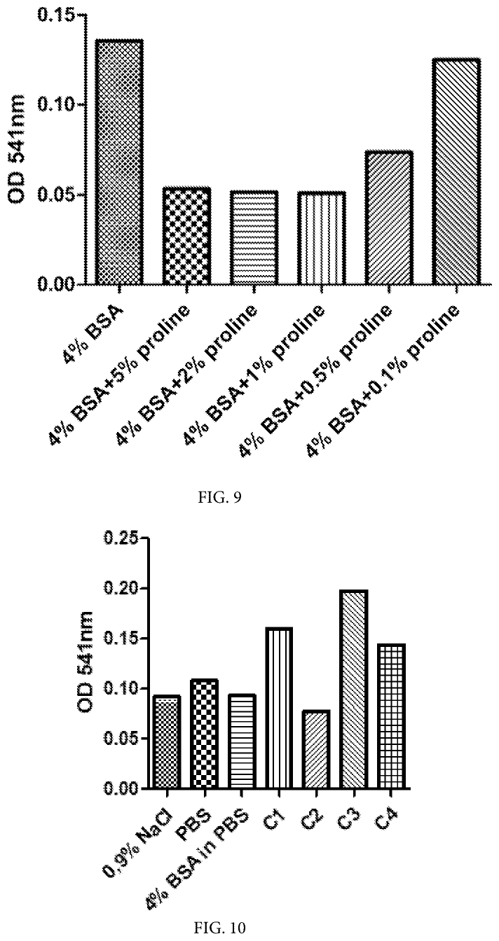

In conventional cryopreservation of RBCs, the deglycerolization process has adverse effects on the final quality of cells. For clinical applications, the efficiency of resuspension of frozen-thawed cells must be considered in order to obtain a highly concentrated cell suspension equivalent to the physiological cell density. In FIG. 7, no hemolysis (absorbance at 541 nm less than 0.005) occurred after resuspension of thawed cells (BSA=0.28, at 100 μm·s⁻¹) compared to the positive control, i.e. complete hemolysis of RBCs. In other words, the cells can be cryopreserved in a relatively dilute state, but once needed, these cells could be thawed, concentrated and then mixed in specific solutions without time-consuming and harmful post-purification of the cells, or also subjected to simple centrifugation, which is a substantial advantage of the method of the disclosure.

Further analysis of esterase-based metabolic activity indicated that more than 95% of RBCs preserved after freezing and thawing are alive, regardless of the experimental conditions. Regarding viability and quality of RBCs, the 0.28 BSA volume fraction group showed good cryosurvival and resulted in a cell size similar to that of the fresh cells. The comparable recovery and viability of RBCs thawed after 1 day and 106 days of storage made it possible to extend the commonly accepted shelf life of RBCs from 49 days to 106 days. Considering the physiological cell density, the low hemolysis rate during resuspension of thawed cells provides a simple tool for enriching RBCs for transfusion.

1. A composition comprising non-adherent cells and an isotonic aqueous solution comprising at least one non-toxic cryoprotectant at a concentration of between 4 and 50% by weight/volume (w/v) and selected from albumins.

2. The composition according to claim 1, wherein the cryoprotectant is human serum albumin.

3. The composition according to claim 1, wherein it further comprises a cryoprotective additive selected from sucrose at a concentration of between 0.5 and 5% w/v, trehalose at a concentration of between 0.5 and 5% w/v, glucose at a concentration of between 0.5 and 5% w/v and carboxymethyl cellulose at a concentration of between 0.1 and 1% w/v.

4. The composition according to claim 1, wherein the non-adherent cells are blood cells, preferably red blood cells.

5. A method for preserving non-adherent cells by freezing comprising the steps of:
a) suspending said non-adherent cells in a cryopreservation medium composed of an isotonic aqueous solution with at least one non-toxic cryoprotectant at a concentration of between 4 and 50% by weight and selected from albumins;
b) optionally, conditioning the mixture obtained in step a);
c) directional freezing of the mixture obtained in step a); and
d) preservation at a temperature between −79° C. and −197° C., preferably at approximately −80° C.

6. The method for preserving non-adherent cells by freezing according to claim 5, wherein the cryoprotectant is human serum albumin.

7. The method for preserving non-adherent cells by freezing according to claim 5, wherein the cryopreservation medium further comprises a cryoprotective additive selected from sucrose, glucose, trehalose and carboxymethyl cellulose.

8. The method for preserving non-adherent cells by freezing according to claim 5, further comprising thawing the non-adherent cells comprising the steps of:
i. contacting the frozen cells with a surface heated to a temperature of between 35 and 45° C.;
ii. centrifugation; and
iii. suspending the cells from the centrifugation supernatant in a saline solution comprising between 1 and 10% proline.

9. A kit for preserving non-adherent cells comprising at least one sealed device for storing sterile biological fluids and an isotonic aqueous solution comprising at least one non-toxic cryoprotectant at a concentration of between 4 and 50% by weight and selected from albumins and, optionally, a cryoprotective additive such as sucrose, carboxymethyl cellulose, trehalose or glucose.

10. The composition according to claim 2, wherein it further comprises a cryoprotective additive selected from sucrose at a concentration of between 0.5 and 5% w/v, trehalose at a concentration of between 0.5 and 5% w/v, glucose at a concentration of between 0.5 and 5% w/v and carboxymethyl cellulose at a concentration of between 0.1 and 1% w/v.

11. The composition according to claim 2, wherein the non-adherent cells are blood cells, preferably red blood cells.

12. The composition according to claim 3, wherein the non-adherent cells are blood cells, preferably red blood cells.

13. The method for preserving non-adherent cells by freezing according to claim 6, wherein the cryopreservation medium further comprises a cryoprotective additive selected from sucrose, glucose, trehalose and carboxymethyl cellulose.

14. The method for preserving non-adherent cells by freezing according to claim 6, further comprising thawing the non-adherent cells comprising the steps of:
i. contacting the frozen cells with a surface heated to a temperature of between 35 and 45° C.;
ii. centrifugation; and
iii. suspending the cells from the centrifugation supernatant in a saline solution comprising between 1 and 10% proline.

15. The method for preserving non-adherent cells by freezing according to claim 7, further comprising thawing the non-adherent cells comprising the steps of:
i. contacting the frozen cells with a surface heated to a temperature of between 35 and 45° C.;
ii. centrifugation; and
iii. suspending the cells from the centrifugation supernatant in a saline solution comprising between 1 and 10% proline.
